(19) United States
(12) Patent Application Publication
(10) Pub. No.: US 2009/0087921 A1
(43) Pub. Date: Apr. 2, 2009

(54) VAPORIZED HYDROGEN PEROXIDE CONCENTRATION DETECTOR

(75) Inventor: Aaron L. Hill, Erie, PA (US)

Correspondence Address: KUSNER & JAFFE;HIGHLAND PLACE SUITE 310, 6151 WILSON MILLS ROAD, HIGHLAND HEIGHTS, OH, 44143, US

(73) Assignee: STERIS Inc.

(21) Appl. No.: 11/865,786

(22) Filed: Oct. 2, 2007

(51) Int. Cl. G01N 33/00; A61L 2/20

(52) U.S. Cl. 436/135; 422/30

(57) ABSTRACT

A method of determining the presence of vaporized hydrogen peroxide (VHP) in a region, comprising the steps of providing a sealable region having an inlet port and an outlet port, creating a flow of a carrier gas into, through and out of the region, delivering vaporized hydrogen peroxide into the carrier gas flow upstream of the region inlet port, destroying the vaporized hydrogen peroxide at a first location downstream from the region outlet port, monitoring the temperature of the carrier gas before and after the first location, and determining a presence of vaporized hydrogen peroxide in the region based upon the temperature readings before and after the first location.

### FIELD OF THE INVENTION

The present invention relates generally to the art of sterilization and decontamination, and more particularly to a system for determining the concentration of a gaseous or vapor phase sterilant in a sterilization or decontamination system.

### BACKGROUND OF THE INVENTION

Sterilization methods are used in a broad range of applications, and have used an equally broad range of sterilization agents. As used herein the term “sterilization” refers to the inactivation of all bio-contamination, especially on inanimate objects. The term “disinfectant” refers to the inactivation of organisms considered pathogenic.

Gaseous and vapor sterilization/decontamination systems rely on maintaining certain process parameters in order to achieve a target sterility or decontamination assurance level. For hydrogen peroxide vapor sterilization/decontamination systems, those parameters include the concentration of the hydrogen peroxide vapor, the degree of saturation, the temperature and pressure and the exposure time. By controlling these parameters, the desired sterility assurance levels can be successfully obtained while avoiding condensation of the hydrogen peroxide due to vapor saturation.

Because of the potential for degradation of the sterilant, monitoring the hydrogen peroxide concentration within a sterilization or decontamination chamber is important to ascertain whether sufficient sterilant concentration is maintained long enough to effect sterilization of objects within the chamber.

To insure the flow of hydrogen peroxide to the vaporizer, it has been known to use pressure switches to measure the static pressure head of the hydrogen peroxide solution in the injection lines to a vaporizer to insure there is sterilant in the injection lines. Some systems utilize a balance to measure the actual mass of the sterilant being injected into a vaporizer. In systems where pressure switches are used, the static head pressure may be reduced when a vacuum is created in the deactivation chamber. This vacuum may cause the pressure switch to generate a false “no sterilant” alarm. In cases where a balance is used to measure sterilant flow, there is no guarantee that the sterilant is actually making it to the vaporizer. Broken lines or disconnected tubing between the balance and the vaporizer can lead to false belief of sterilant in the decontamination chamber. Still further, any system, like the aforementioned pressure switches or balances, that precedes the vaporizer cannot detect or insure that the sterilant actually reaches the decontamination chamber.

It has also been known to detect the presence of vaporized hydrogen peroxide (VHP) in a chamber by means of chemical or biological indicators. Biological indicators, however, must be incubated for several days before knowing if sterilant is present, and chemical indicators generally provide a visual indication (typically by changing colors), thereby requiring operator intervention to abort a sterilization/decontamination cycle if the chemical indicators do not provide a positive indication of the presence of the sterilant. Another shortcoming of biological and chemical indicators is that they can only provide an indication of the presence of vaporized hydrogen peroxide (VHP), but cannot provide an indication of the amount of vaporized hydrogen peroxide (VHP) present.

It has been proposed to use infrared (IR) sensors to determine the actual vaporized hydrogen peroxide (VHP) concentration present. IR sensors are expensive, delicate and bulky, making accurate vaporized hydrogen peroxide (VHP) measurements difficult. Such sensors require frequent calibration and seem to require frequent lamp change-outs when used for high-concentration vaporized hydrogen peroxide (VHP) measurements. In this respect, it is desirable that measurements be made in real time as a sterilization process proceeds.

The present invention overcomes these and other problems, and provides a system for detecting concentrations of vapor hydrogen peroxide in a sterilization/deactivation chamber.

### SUMMARY OF THE INVENTION

In accordance with the present invention, there is provided a method of determining the presence of vaporized hydrogen peroxide (VHP) in a region, comprising the steps of:

providing a sealable region having an inlet port and an outlet port, and a closed loop conduit having a first end fluidly connected to the region inlet port and a second end fluidly connected to the region outlet port;

re-circulating a flow of a carrier gas into, through and out of the region and around the closed loop conduit;

delivering vaporized hydrogen peroxide into the re-circulating carrier gas flow upstream of the region inlet port;

destroying the vaporized hydrogen peroxide at a first location downstream from the region outlet port;

monitoring the temperature of the carrier gas before and after the first location; and

determining a presence of vaporized hydrogen peroxide in the region based upon the temperature of the carrier gas before and after the first location.

In accordance with another aspect of the present invention, there is provided a closed loop, flow through method of vapor phase decontamination in a sealable chamber or region having an inlet port and an outlet port, and a closed loop conduit fluidly connecting the outlet port to the inlet port, the method comprising the steps of:

re-circulating a flow of a carrier gas into, through and out of the chamber, and through the closed loop conduit;

supplying vaporized hydrogen peroxide into the re-circulating carrier gas flow;

destroying the vaporized hydrogen peroxide to form water and oxygen at a first location downstream from the outlet port;

monitoring the temperature of the carrier gas before and after the first location; and

estimating the concentration of vaporized hydrogen peroxide in the region based upon the temperature of the carrier gas before and after the first location.

In accordance with yet another aspect of the present invention, there is provided a method of determining the presence of vaporized hydrogen peroxide (VHP) in a region, comprising the steps of:

providing a sealable region having an inlet port and an outlet port;

creating a flow of a carrier gas into, through and out of the region;

delivering vaporized hydrogen peroxide into the carrier gas flow upstream of the region inlet port;

destroying the vaporized hydrogen peroxide at a first location downstream from the region outlet port;

monitoring the temperature of the carrier gas before and after the first location; and

determining a presence of vaporized hydrogen peroxide in the region based upon the temperature readings before and after said first location.

An advantage of the present invention is a system for determining the concentration of vaporized hydrogen peroxide in an enclosed chamber.

Another advantage of the present invention is a sensor as described above that can determine the concentration of vaporized hydrogen peroxide during the course of a deactivation cycle.

Another advantage of the present invention is a sensor as described above that does not require operator intervention.

These and other advantages will become apparent from the following description of a preferred embodiment taken together with the accompanying drawings and the appended claims.

### BRIEF DESCRIPTION OF THE DRAWINGS

The invention may take physical form in certain parts and arrangement of parts, a preferred embodiment of which will be described in detail in the specification and illustrated in the accompanying drawings which form a part hereof, and wherein:

FIG. 1 is a schematic view of a vapor hydrogen peroxide deactivation system.

### DETAILED DESCRIPTION OF PREFERRED EMBODIMENT

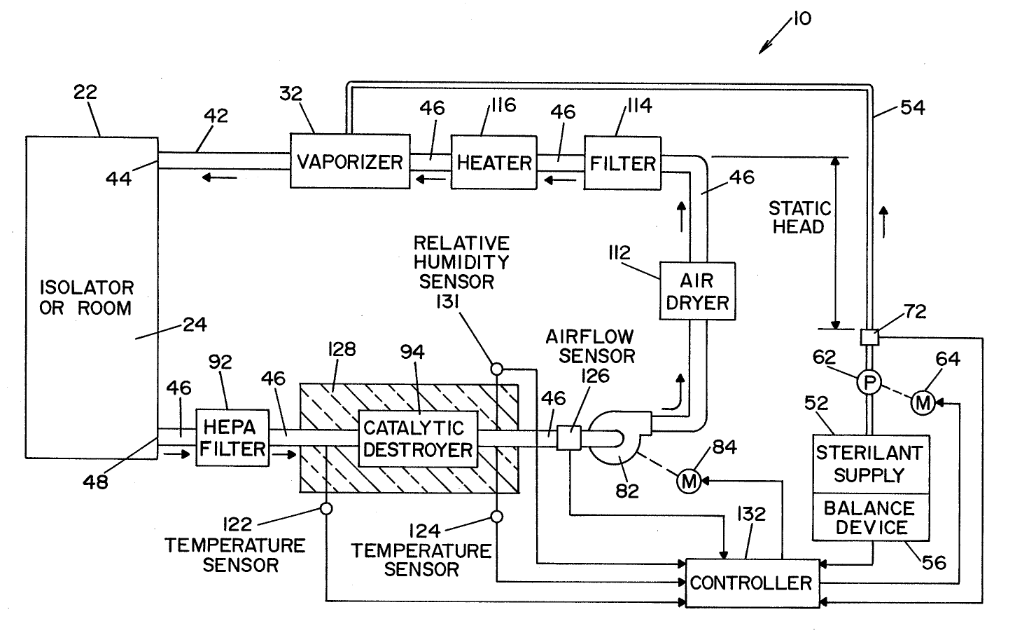

Referring now to the drawings wherein the showings are for the purpose of illustrating a preferred embodiment of the invention only, and not for the purpose of limiting same, FIG. 1 shows a vaporized hydrogen peroxide sterilization system 10, illustrating a preferred embodiment of the present invention. System 10 includes means operable to determine the presence and/or concentration of vaporized hydrogen peroxide, i.e., a two-component, vapor-phase sterilant, and will be described with particular reference thereto. It will of course be appreciated that the invention may find advantageous application in determining the concentration of other multi-component, vapor-phase sterilants.

In the embodiment shown, system 10 includes an isolator or room 22 that defines an inner sterilization/decontamination chamber or region 24. It is contemplated that articles to be sterilized or decontaminated may be disposed within isolator or room 22. A vaporizer 32 (also referred to herein as generator) is connected to sterilization/decontamination chamber or region 24 of room or isolator 22 by means of a supply conduit 42. Supply conduit 42 defines a vaporized hydrogen peroxide (VHP) inlet 44 to chamber or region 24. Vaporizer 32 is connected to a liquid sterilant supply 52 by a feed line 54. A conventionally known balance device 56 is associated with sterilant supply 52, to measure the actual mass of sterilant being supplied to vaporizer 32.

A pump 62 driven by a motor 64 is provided to convey metered amounts of the liquid sterilant to vaporizer 32 where the sterilant is vaporized by conventionally known means. In an alternate embodiment, pump 62 is provided with an encoder (not shown) that allows monitoring of the amount of sterilant being metered to vaporizer 32. If an encoder is provided with pump 62, balance device 56 is not required. If the balance is not used, a pressure switch 72 is provided in the feed line to indicate the presence of sterilant. Pressure switch 72 is operable to provide an electrical signal in the event that a certain static head pressure, normally produced by the presence of the sterilant, does not exist in feed line 54.

Isolator or room 22 and vaporizer 32 are part of a closed loop system that includes a return conduit 46 that connects isolator or room 22 (and sterilization/decontamination chamber or region 24) to vaporizer 32. Return conduit 46 defines a VHP return 48 from the sterilization/decontamination chamber or region 24. A blower 82, driven by a motor 84, is disposed within return conduit 46 between isolator or room 22 and vaporizer 32. Blower 82 is operable to circulate sterilant and air through the closed loop system. A first filter 92 and catalytic destroyer 94 are disposed in return conduit 46 between blower 82 and isolator or room 22, as illustrated in FIG. 1. First filter 92 is preferably a HEPA filter and is provided to remove contaminants flowing through system 10. Catalytic destroyer 94 is operable to destroy hydrogen peroxide (H₂O₂) flowing therethrough, as is conventionally known. Catalytic destroyer 94 converts the hydrogen peroxide (H₂O₂) into water and oxygen. An air dryer 112, second filter 114 and heater 116 are disposed within return conduit 46 between blower 82 and vaporizer 32. Air dryer 112 is operable to remove moisture from air blown through the closed loop system. Second filter 114 is operable to filter the air blown through return conduit 46 by blower 82. Heater 116 is operable to heat air blown through return conduit 46 by blower 82. In this respect, air is heated prior to the air entering vaporizer 32.

A first temperature sensor 122 is disposed within return conduit 46 upstream, i.e., before, catalytic destroyer 94. As shown in the drawing, first temperature sensor 122 is disposed between first filter 92 and catalytic destroyer 94. A second temperature sensor 124 is disposed within return conduit 46 at a location downstream, i.e. beyond, catalytic destroyer 94. As showing in the drawing, second temperature sensor 124 is disposed between blower 82 and catalytic destroyer 94. An airflow sensor 126 is disposed in return conduit 46 between blower 82 and catalytic destroyer 94. A relative humidity sensor 131 is disposed in return conduit 46 at a location downstream, i.e., beyond catalytic destroyer 94. Relative humidity sensor 131 is preferably disposed at the same location as second temperature sensor 124. Temperature sensors 122 and 124 are operable to the sense temperature of the carrier gas flowing through return conduit 46 at locations before (i.e. upstream of) and beyond (i.e., downstream from) catalytic destroyer 94. Airflow sensor 126 is operable to sense the flow of carrier gas through return conduit 46. Return conduit, at least in the area of catalytic destroyer, is preferably insulated, as schematically illustrated in the drawing wherein insulation 128 is shown surrounding catalytic destroyer 94 and portions of return conduit 46.

First temperature sensor 122, second temperature sensor 124 and airflow sensor 126 provide electrical signals to a system controller 132 that is schematically illustrated in FIG. 1. Controller 132 is a system microprocessor or microcontroller programmed to control the operation of system 10. As illustrated in FIG. 1, controller 132 is also connected to motors 64, 84, pressure switch 72 and balance device 56.

The present invention shall now be further described with reference to the operation of system 10. A typical sterilization/decontamination cycle includes a drying phase, a conditioning phase, a decontamination phase and an aeration phase. Prior to running a sterilization/decontamination cycle, data regarding the percent of hydrogen peroxide in the sterilant solution is entered, i.e., inputted, into controller 132. As noted above, in a preferred embodiment a sterilant solution of 35% hydrogen peroxide and 65% water is used. However, other concentrations of hydrogen peroxide and water are contemplated.

Isolator or room 22, supply conduit 42 and return conduit 46 define a closed loop conduit circuit. When a sterilization/decontamination cycle is first initiated, controller 132 causes blower motor 84 to drive blower 82, thereby causing a carrier gas to circulate through the closed loop circuit. During a drying phase, vaporizer 32 is not operating. Air dryer 112 removes moisture from the air circulating through the closed loop system, i.e., through supply conduit 42, return conduit 46 and sterilization/decontamination chamber or region 24 or isolator or room 22, as illustrated by the arrows in FIG. 1. When the air has been dried to a sufficiently low humidity level, the drying phase is complete.

The conditioning phase is then initiated by activating vaporizer 32 and sterilant supply motor 64 to provide sterilant to vaporizer 32. In a preferred embodiment of the present invention, the sterilant is a hydrogen peroxide solution comprised of about 35% hydrogen peroxide and about 65% water. A sterilant solution comprised of different ratios of hydrogen peroxide is also contemplated. Within vaporizer 32, the liquid sterilant is vaporized to produce vaporized hydrogen peroxide (VHP) and water vapor, in a conventionally known manner. The vaporized sterilant is introduced into the closed loop conduit circuit and is conveyed through supply conduit 42 by the carrier gas (air) into sterilization/decontamination chamber or region 24 within isolator or room 22. During the conditioning phase, VHP is injected into sterilization/decontamination chamber or region 24 at a relatively high rate to bring the hydrogen peroxide level up to a desired level in a short period of time. During the conditioning phase, blower 82 causes air to continuously circulate through the closed loop system. As VHP enters chamber or region 24 from vaporizer 32, VHP is also being drawn out of chamber or region 24 through catalytic destroyer 94 where it is broken down into water and oxygen.

After the conditioning phase is completed, the decontamination phase is initiated. During the decontamination phase, the sterilant injection rate to vaporizer 32 and to sterilization/decontamination chamber or region 24 is decreased to maintain the hydrogen peroxide concentration constant at a desired level. The decontamination phase is run for a predetermined period of time, preferably with the hydrogen peroxide concentration remaining constant at a desired level, for a predetermined period of time that is sufficient to effect the desired sterilization or decontamination of sterilization/decontamination chamber or region 24, and items therein.

After the decontamination phase is completed, controller 132 causes vaporizer 32 to shut down, thereby shutting off the flow of vaporized hydrogen peroxide (VHP) into sterilization/decontamination chamber or region 24.

Thereafter, the aeration phase is run to bring the hydrogen peroxide level down to an allowable threshold (about 1 ppm). In this respect, as will be appreciated, blower 82 continues to circulate the air and sterilant through the closed loop system, thereby causing the last of the vaporized hydrogen peroxide (VHP) to be broken down by catalytic destroyer 94.

Throughout the respective operational phases, first and second temperature sensors 122 and 124 monitor the temperature, within return conduit 46, at locations upstream (before) and downstream (after) of catalytic destroyer 94, and provide electrical signals indicative of the temperatures within return conduit 46 to controller 132.

In accordance with the present invention, controller 132 is programmed to determine the presence and concentration of VHP within sterilization/decontamination chamber or region 24, based upon the temperature data from first and second sensors 122 and 124. In this respect, during the operation of system 10, air and sterilant flow through a closed loop system, as described above. As VHP exits sterilization/decontamination chamber or region 24, the hydrogen peroxide (H₂O₂) is destroyed in catalytic destroyer 94, where the hydrogen peroxide is broken down into water and oxygen per the following chemical equation:

2H₂O_{2→}2H₂O₊O_{2.}

This process is exothermic which releases heat in the amount of 1,233 BTU/lbm (2.868 KJ/g) of hydrogen peroxide. The heat generated within system 10 will be dependant on concentration of hydrogen peroxide blown through destroyer 94. Assuming that all the heat generated in this reaction goes into the air stream (this will occur once destroyer 94 reaches the steady state temperature if destroyer 94 is insulated which will keep down the heat loss through the walls of destroyer 94), the peroxide concentration can be calculated using the air temperature increase through destroyer 94.

Thus, during the conditioning phase and the decontamination phase, any sensed temperature difference between first temperature sensor 122 and second temperature sensor 124 is a product of the breakdown of vaporized hydrogen peroxide (VHP) and water vapor introduced by vaporizer 32. Controller 132 is programmed to monitor the temperature changes, and to calculate an estimated concentration of hydrogen peroxide. Since blower 82 continuously circulates air and sterilant through the closed loop system, the calculations of hydrogen peroxide concentration, that are based upon the temperatures in return conduit 46, represent the amount of hydrogen peroxide within sterilization/decontamination chamber or region 24 prior to passing through catalytic destroyer 94.

The present invention is based upon the assumption that the time rate of change of heat expelled by the breakdown of peroxide (\dot{Q}_{P}) is equal to the time rate of change of heat absorbed by the air stream in the system (\dot{Q}_{A}). In other words,

\dot{Q}_{P}=\dot{Q}_{A} (1)

It is believed that the heat expelled by the breakdown of peroxide (\dot{Q}_{P}) is determined by the following equation:

\dot{Q}_{P}=C_{H}·H·F [expressed in (BTU/min) or (KJ/min)] (2)

where:
C_{H}=Hydrogen Peroxide concentration in air stream [expressed in (lbm/ft³) or (gram/liter)]
F=Air flow rate [expressed in (standard ft³/min) or (standard liter/min)]
H=Heat of exothermic reaction of peroxide breakdown, i.e., 1,233 BTU/lbm or (2.868 KJ/gram)

It is believed that the heat absorbed by the air stream (\dot{Q}_{A}) is determined by the following equation:

\dot{Q}_{A}=\dot{m}_{A}·C_{P}·ΔT [expressed in (BTU/min) or (KJ/min)] (3)

where:
\dot{m}_{Air}=Mass flow of air [expressed in (lbm/min) or (g/min])
ΔT=Change in air temperature through the destroyer [expressed in (° F.) or (° C.)]
C_{P}=Specific heat of moist air

The mass flow of air \dot{m}_{Air} is equal to the air flow rate (F) times the density (ρ) of standard air. The density (ρ) of standard air is approximately 0.075 lbm/ft³ or 1.201 g/liter.

It is believed that the specific heat (C_{P}) of moist air is determined by the following equation:

C_{P}=(0.24+0.45ω) BTU/lbm-° F. [(0.001+0.00188 ω)KJ/kg-° C.] (4)

where:
ω=Humidity ratio of air stream (mass of water divided by mass of dry air)

The humidity ratio is calculated using a temperature, T, and a relative humidity, RH, determined at a point beyond catalytic destroyer 94, as shown in the drawings.

The following equation is used to convert the relative humidity, RH, into absolute humidity:

RH={1+0.622/ωₛ}/{1+0.622/ω} (5)

where:
RH=Relative humidity
ωₛ=The humidity ratio at saturation (mass of water/mass of air)
ω=The humidity ratio at the given temperature and RH

Solving for ω results in the following equation:

$$ω = \frac{( 0.622 ) ( RH ) ( {ω}_{s} )}{{ω}_{s} + 0.622 - ( RH ) ( {ω}_{s} )} \qquad ( 6 )$$

The saturated humidity ratio is calculated using the following equation:

$${ω}_{s} = 0.622 \frac{{P}_{ω , s}}{P - {P}_{ω , s}} \qquad ( 7 )$$

where:
P_{ω,s}=Vapor pressure of water at temperature given below [expressed in (psi) or (kpascal)]
P=Atmospheric pressure [expressed in (psi) or (pascal)]

For temperatures above 32° F. (0° C.), the vapor pressure of water at saturation (psi) (kpascal) is determined by the following equation:

$${P}_{ω , s} = K \{ exp ( C 8 / ( TF + 460 ) + C 9 + ( C 10 ) ( TF + 460 ) + 2 ( C 11 ) ( TF + 460 ) + 3 ( C 12 ) ( TF + 460 ) + ( C 13 ) [ log ( TF + 460 ) ] \} \qquad ( 8 )$$

where:
K=1.0 for psi or 6.894 for kpascal:
TF=Vapor temperature (° F.) or (° C.*1.8-32)
C8=10440.397
C9=11.29465
C10=0.027022355
C11=0.00001289036
C12=2.4780681E-09
C13=6.5459673

Substituting equations (2) and (3) in equation (1) results in the following equation:

C_{H}·H·F=\dot{m}_{Air}·Cₚ·ΔT (9)

Solving equation (9) for the concentration of hydrogen peroxide (C_{H}) results in the following equation:

$${C}_{H} = \frac{{\dot{m}}_{Air} \cdot {C}_{p} \cdot Δ T}{H \cdot F} ( lbm \text{/} {ft}^{3} ) or ( gram \text{/} liter ) \qquad ( 10 )$$

The foregoing calculations are further illustrated by way of example.

A typical VHP® cycle has an air flow of about 20 scfm (566.4 liters/min) and a peroxide concentration of about 1 mg/liter (6.243×10⁻⁵ lbm/ft³), or (0.001 g/liter) of hydrogen peroxide sterilant and 1.857 mg/liter (1.159×10⁻⁵ lbm/ft³), or (0.001857 g/liter) of water (based on 35% H₂O₂). The given water concentration equates to a humidity ratio of 0.0036 at a temperature of 77° F. (25° C.). Solving equation (10) for ΔT gives 4.2° F. (2.3° C.) which is well within the accuracy of currently available temperature measurement devices (RTD's, thermocouples etc.).

In reality, some heat will be lost through conduction and convection from the destroyer which will affect the magnitude of the measure ΔT. To account for this, a calibration may be run using a known standard, such as near IR instruments, for measuring the hydrogen peroxide and producing a calibration curve that can account for external heat losses.

In most cases, with smaller enclosures, the reduction in H₂O₂ concentration due to the half-life of the H₂O₂ does not significantly affect the hydrogen peroxide level. In large enclosures or rooms where the H₂O₂ resides for long periods of time and comes in contact with catalytic substances, consideration must be given to the reduction in H₂O₂ concentration due to the half-life.

In accordance with another aspect of the present invention, controller 132 is operable to monitor the temperatures in return conduit 46 to make sure the temperature difference increases at a desired rate during the conditioning phase, or remains relatively stable during the decontamination phase. If controller 132 determines that the temperature difference is not increasing (during the conditioning phase) or does not remain stable during the decontamination phase, an error indication is provided. For example, the operator may be provided with a visual display, such as “out of sterilant” or “check for leaks,” or an alarm may also sound indicating an improper sterilization cycle.

Controller 132 can calculate the amount of vaporized hydrogen peroxide (VHP) that was within sterilization/decontamination chamber or region 24 based upon the foregoing equations. As indicated above, during the decontamination phase, the temperature difference sensors 122 and 124 should remain fairly constant as the amount of vaporized hydrogen peroxide (VHP) is maintained at the constant, desired level. Following the completion of the decontamination phase, the aeration phase reduces the amount of VHP in system 10 as blower 82 continuously circulates air and sterilant through system 10 until catalytic destroyer 94 has broken down the VHP, and air dryer 112 eventually removes the moisture from system 10.

The present invention thus provides a simple yet efficient method of determining the presence and concentration of vaporized hydrogen peroxide within sterilization/decontamination chamber or region 24 by monitoring the endothermic process resulting from the breakdown of the components of the vaporized hydrogen peroxide.

The foregoing description is a specific embodiment of the present invention. It should be appreciated that this embodiment is described for purposes of illustration only, and that numerous alterations and modifications may be practiced by those skilled in the art without departing from the spirit and scope of the invention. It is intended that all such modifications and alterations be included insofar as they come within the scope of the invention as claimed or the equivalents thereof.

Having described the invention, the following is claimed:

1. A method of determining the presence of vaporized hydrogen peroxide (VHP) in a region, comprising the steps of:
providing a sealable region having an inlet port and an outlet port, and a closed loop conduit having a first end fluidly connected to the region inlet port and a second end fluidly connected to the region outlet port;
re-circulating a flow of a carrier gas into, through and out of said region and around the closed loop conduit;
delivering vaporized hydrogen peroxide into the re-circulating carrier gas flow upstream of the region inlet port;
destroying the vaporized hydrogen peroxide at a first location downstream from the region outlet port;
monitoring the temperature of said carrier gas in said system before and after said first location; and
determining a presence of vaporized hydrogen peroxide in said region based upon the temperature readings before and after said first location.

2. A method as defined in claim 1, wherein said carrier gas is air.

3. A method as defined in claim 1, wherein said destroying step includes catalytically decomposing the hydrogen peroxide vapor into water and oxygen.

4. A closed loop, flow through method of vapor phase decontamination in a sealable chamber or region having an inlet port and an outlet port, and a closed loop conduit fluidly connecting the outlet port to the inlet port, the method comprising the steps of:
re-circulating a flow of a carrier gas into, through and out of the chamber, and through the closed loop conduit;
supplying vaporized hydrogen peroxide into the re-circulating carrier gas flow;
destroying the vaporized hydrogen peroxide to form water and oxygen at a first location downstream from said outlet port;
monitoring the temperature of said carrier gas before and after said first location; and
estimating the concentration of vaporized hydrogen peroxide in said region based upon the temperature of said carrier gas before and after said first location.

5. A closed loop, flow through method as defined in claim 4, wherein said carrier gas is air.

6. A closed loop, flow through method as defined in claim 4, wherein said destroying step includes catalytically decomposing the hydrogen peroxide vapor into water and oxygen.

7. A method of determining the presence of vaporized hydrogen peroxide (VHP) in a region, comprising the steps of:
providing a sealable region having an inlet port and an outlet port;
creating a flow of a carrier gas into, through and out of said region;
delivering vaporized hydrogen peroxide into the carrier gas upstream of the region inlet port;
destroying the vaporized hydrogen peroxide at a first location downstream from the region outlet port;
monitoring the temperature of said carrier gas before and after said first location; and
determining a presence of vaporized hydrogen peroxide in said region based upon the temperature readings before and after said first location.

8. A method as defined in claim 7, wherein said carrier gas is air.

9. A method as defined in claim 7, wherein said destroying step includes catalytically decomposing the hydrogen peroxide vapor into water and oxygen.

10. A method as defined in claim 7, wherein said sealed region is part of a closed loop system having a closed loop conduit, said closed loop conduit having a first end fluidly connected to the region inlet port and a second end fluidly connected to the region outlet port.
